(19) United States
(12) Patent Application Publication
(10) Pub. No.: US 2010/0063579 A1
(43) Pub. Date: Mar. 11, 2010

(54) STENT

(75) Inventor: Sung-Soon An, Seoul (KR)

Correspondence Address: SHERIDAN ROSS PC, 1560 BROADWAY, SUITE 1200, DENVER, CO, 80202, US

(73) Assignee: STANDARD SCI-TECH INC., Seoul (KR)

(21) Appl. No.: 12/516,124

(22) PCT Filed: Sep. 28, 2007
(86) PCT No.: PCT/KR2007/004731

§ 371 Date: Jul. 20, 2009

(30) Foreign Application Priority Data
Nov. 22, 2006 (KR) 10-2006-0115557

(51) Int. Cl. A61F 2/82

(52) U.S. Cl. 623/1.15; 623/1.42

(57) ABSTRACT

The present invention discloses a stent that is designed to be easily removed from a lumen such as an esophagus, a blood vessel, or other organs of a human body without performing a surgical operation. The stent of the present invention includes an elastic body unit that is formed in a cylindrical shape and has an elastic force and a covering unit that is combined with or coated on the elastic body unit. The covering unit includes an extending portion that is separated from an end portion of the elastic body unit.

### TECHNICAL FIELD

The present invention relates to a stent. More particularly, the present invention relates to a stent that is designed to be easily removed from a lumen, such as a blood vessel or other organs of a human body, without performing a surgical operation.

### BACKGROUND ART

When a bodily fluid or blood cannot effectively flow along a blood vessel, a gastrointestinal tract, a biliary tract, and the like due to a malignant or benign disease occurring in the tracts or blood vessel, a stent is used to normalize the flow of the bodily fluid or blood by being inserted in a narrowed or blocked portion of the tracts or blood vessel under X-ray radiation. As shown in FIG. 6, the stent includes an elastic body unit 101 and a covering unit 103 covering the elastic body unit 101. The elastic body unit 101 is formed in a cylindrical shape by thin, long elastic members connected to each other. In order to enlarge the narrowed lumen, the elastic body unit 101 is shrinkable and expandable in a radial direction of the cylindrical elastic body unit 101. The covering unit 103 is formed by coating or combining a synthetic resin material on or with the elastic body unit 101 when there is a need to make the stent function as the lumen of the human body or to prevent the grown lesion from infiltrating inside the stent. Particularly, in order to easily remove the stent from the lumen of the human body, a connecting string is provided to an end of the elastic body unit 101. The connecting string may be disposed to interconnect end portions of the elastic body unit 101. When a portion of the connecting string 105 is pulled by a stent removing device, the connecting string 105 is designed to converge the end portions of the elastic body unit 101 toward the central axis of the cylindrical stent. Therefore, the stent is shrunk to have a reduced diameter and thus easily removed from the lumen by the stent removing device.

The connecting string 105 that functions to shrink the stent to the relatively small diameter penetrates the covering unit 103 at a portion where it is connected to the elastic body unit 101. That is, since the elastic body unit 101 and the covering unit 103 are integrally combined with each other at an end portion of the stent, a through hole 103a is provided in the covering unit 103 for the connection of the connecting string 105 to the elastic body unit 101. According to the prior art, when the stent is inserted in the lumen of the human body, the lumen or lesion such as cancer may be grown or transformed and thus penetrate into the covering unit 103 through the through hole 103a that is formed for the connecting string 105. Therefore, when there is a need to remove the stent, a surgical operation is required or the stent cannot be easily removed.

The above information disclosed in this Background section is only for enhancement of understanding of the background of the invention and therefore it may contain information that does not form the prior art that is already known in this country to a person of ordinary skill in the art.

### DISCLOSURE

### Technical Problem

The present invention has been made in an effort to solve the above-described problems. An object of the present invention is to provide a stent that is designed to be easily removed from a lumen of a human body by an improved coupling structure of a covering unit that is coated on or combined with an elastic body unit.

### Technical Solution

Another object of the present invention is to provide a stent that can be easily removed from a lumen of a human body by preventing a lesion that is grown from penetrating through a through hole formed in a covering unit.

To achieve the objects, the present invention provides a stent including an elastic body unit that is formed in a cylindrical shape and has an elastic force and a covering unit that is combined with or coated on the elastic body unit, the covering unit having an extending portion that is separated from an end portion of the elastic body unit.

Preferably, the extending portion may extend over the end portion of the elastic body unit.

Preferably, a connecting string may be coupled to the end portion of the elastic body unit to shrink the elastic body unit.

Preferably, the elastic body unit may be formed by twisting an elastic member having an elastic force in a mesh shape.

Preferably, the covering unit and the extending portion may be formed of a synthetic resin material or a highly polymerized compound.

The present invention also provides a stent including an elastic body unit that is formed in a cylindrical shape and has elastic force, a covering unit combined with the elastic body unit, and a connecting string that is coupled to the end portion of the elastic body unit to shrink the elastic body unit, wherein an outer circumference of the covering unit is coated with a coating layer formed of a lesion growth suppressing material or the covering unit contains a lesion growth suppressing material.

Preferably, the lesion growth suppressing material may include paclitaxel or docetaxel.

### DESCRIPTION OF DRAWINGS

FIG. 1 is a perspective view illustrating a first exemplary embodiment of the present invention.

FIG. 2 is a view taken by viewing FIG. 1 in a direction of an arrow A.

FIG. 3 is a cross-sectional view taken along line III-III of FIG. 1.

FIG. 4 is a perspective view illustrating a second exemplary embodiment of the present invention.

FIG. 5 is a view illustrating a modified example of FIG. 4.

FIG. 6 is a perspective view of a prior art stent.

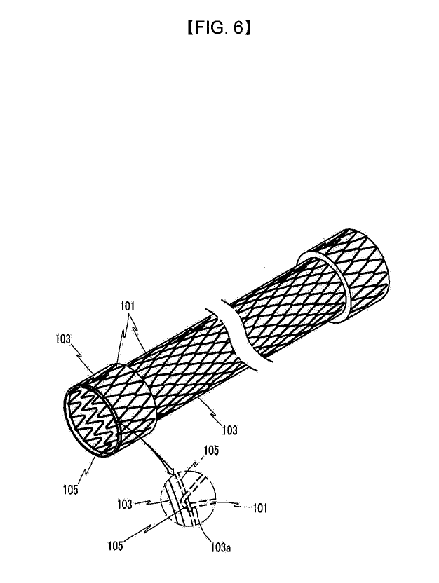

### BEST MODE

Exemplary embodiments of the present invention will be described in detail with reference to the accompanying drawings.

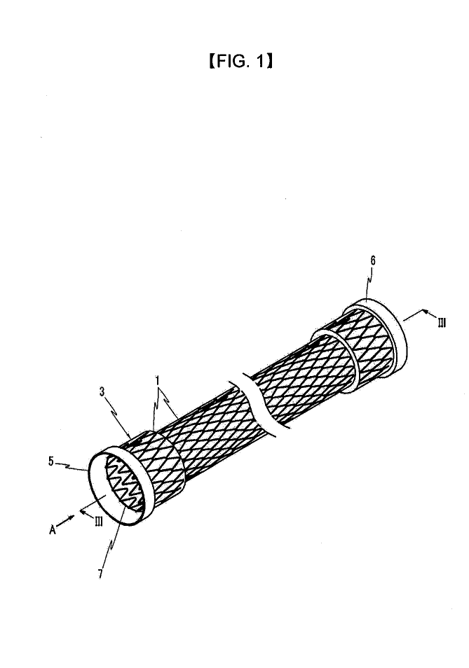

FIG. 1 is a perspective view of a stent according to a first exemplary embodiment of the present invention. The stent of the first exemplary embodiment of the present invention includes a cylindrical elastic body unit 1 and a covering unit 3 coated on or combined with the elastic body unit 1. The elastic body unit 1 is formed in a cylindrical shape by repeatedly twisting a thin, long elastic member with predetermined intervals. However, the elastic body unit 1 is not limited to the above described structure. That is, any structures that can be inserted into a lumen of a human body, such as an esophagus or a blood vessel, to allow bodily fluids to effectively flow along thereof may be applied to the present invention.

The covering unit 3 is formed by coating or combining a relatively soft synthetic resin material on an outer circumference of the elastic body unit 1. The covering unit 3 prevents the lumen or lesion that is grown or transformed from penetrating through apertures of the elastic body unit 1. The covering unit 3 functions as a passage along which bodily fluids can effectively flow when the lumen of the human body is narrowed by the lesion. The covering unit 3 may be formed by coating a synthetic resin material on an outer circumference of the elastic body unit 1 or may be provided in the form of a separate member that is formed of a synthetic resin or a highly polymerized compound and is cylindrical to be inserted around the elastic body unit 1.

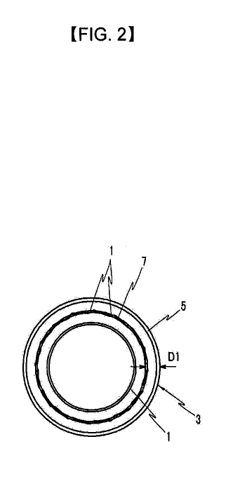

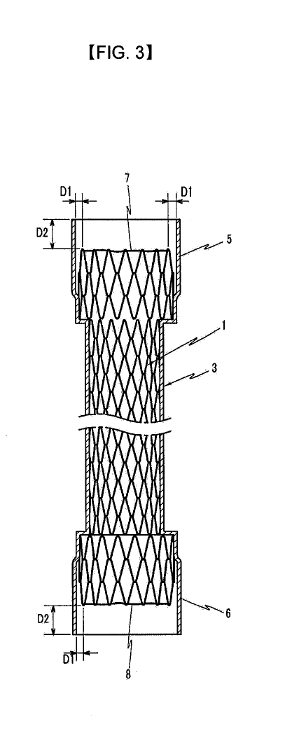

The covering unit 3 includes extending end portions 5 and 6 that extend past corresponding opposite end portions of the elastic body unit 1 by a distance D2 (see FIG. 3). The extending portions 5 and 6 may not be combined with the elastic body unit 1 at partial sections of the opposite end portions of the elastic body unit 1 but disposed around the outer circumferences of the opposite end portions of the elastic body unit 1. That is, as shown in FIG. 2, the extending portion 5 (only one is shown for descriptive convenience) is not combined with the corresponding end portion of the elastic body unit 1 but spaced apart from the corresponding end portion by a predetermined distance D1. In FIGS. 2 and 3, although the extending portion 5 and the corresponding end portion of the elastic body unit 1 are spaced apart from each other by the predetermined interval D1, the present invention is not limited to this exemplary embodiment. That is, as long as the extending end portion 5 is disposed not to be combined with the corresponding end portion of the elastic body unit 1, the object of the present invention will be achieved.

Meanwhile, the end portions of the elastic body unit 1 may be formed by thin, long elastic members that are bent and have valleys and peaks. Connecting strings 7 and 8 (see FIG. 3) are provided on the peaks of the end portions of the elastic body unit 1 to remove the stent out of the lumen by shrinking the stent. The connecting strings 7 and 8 are formed in a conventional structure that is proper for shrinking the elastic body unit 1 and removing the stent using a stent removing device. The connecting strings 7 and 8 are connected only to the elastic body unit 1 but spaced apart from the extending portions 5 and 6 of the covering unit 3. That is, the connecting strings 7 and 8 are structured not to penetrate the covering unit 3. Therefore, no through hole through which the grown lumen or deformed lesion may penetrate into the covering unit 3 is formed in the covering unit 3 and the extending portion 5. In this exemplary embodiment of the present invention, although the stent is designed having opposite end portions that are identically structured, the present invention is not limited to this configuration. That is, the above-described exemplary embodiment may be applied to only one of the opposite end portions of the stent. However, when the extending portions 5 and 6 are identically applied to the respective opposite end portions of the stent, it becomes easy to remove the stent even when the disposed position of the stent is changed.

The stent that is structured as described above according to the exemplary embodiment of the present invention is inserted in a lumen where a lesion is created by a stent insertion device. When the stent is inserted in the lumen for a long time, organic cells around the stent are grown or the lesion is transformed.

According to the exemplary embodiment of the present invention, since there are no holes through which the connecting strings 7 and 8 pass in the extending portions 5 and 6 of the covering unit 3, infiltration of the grown lumen and a transformed lesion inside the covering unit 3 can be prevented. Therefore, when the connecting string 7 or 8 is pulled to remove the stent using the stent removing device, the end portion of the elastic body unit 1 is shrunk, thereby making it easy to remove the stent. In order to realize the stent removal method, a conventional method and device can be identically used. Therefore, according to the exemplary embodiment of the present invention, since the stent can be easily removed without performing a surgical operation, the operation cost can be saved and the patient can quickly recover after the stent is removed.

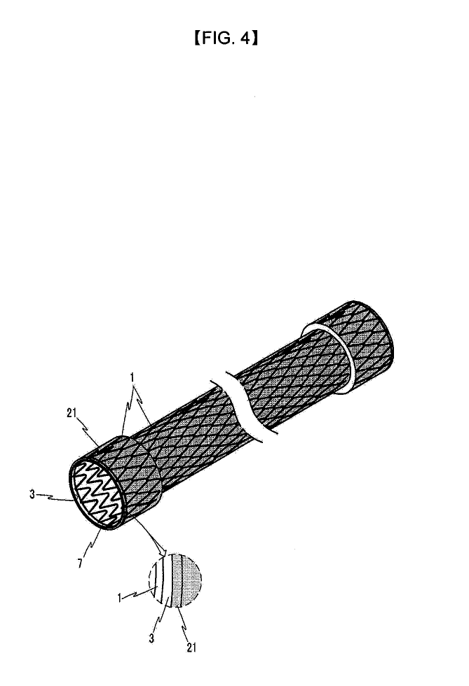

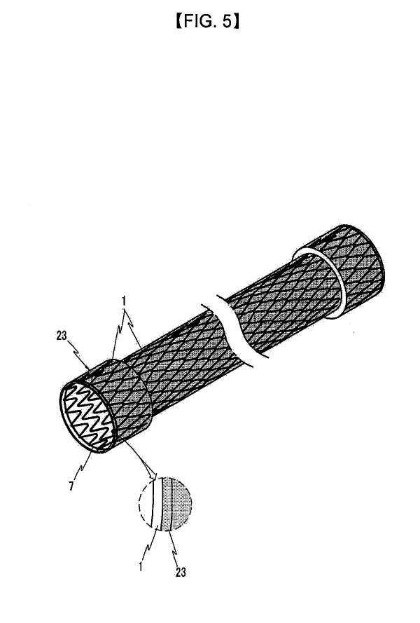

FIG. 4 is a view of a stent according to a second exemplary embodiment of the present invention. Like the stent of the first exemplary embodiment, a stent of a second exemplary embodiment of the present invention includes an elastic body unit 1, a covering unit 3, and a connecting string 7 (only one is shown). The elastic body unit 1, the recovering unit 3, and the connecting string 7 are formed with conventional structures. The recovering unit 3 may be coated with or contain paclitaxel or docetaxel, which is well known as a cancer suppressing material. That is, a cancer suppressing material coating layer 21 may be formed on an outer circumference of the covering unit 3. Alternatively, as shown in FIG. 5, the covering unit may be formed of a material mixed with the cancer suppressing material. That is, the stent of the exemplary embodiment of FIG. 4 further includes the cancer suppressing material coating layer 21 formed on the outer circumference of the covering unit 3. The stent of the exemplary embodiment of FIG. 5 has a covering unit 3 that is formed of a material mixed with the cancer suppressing material. When the stent of the second exemplary embodiment is disposed closely contacting the lesion, the cancer suppressing material pharmacologically acts on a lesion, such as cancer, and so the growth of the lesion can be suppressed. Therefore, according to the stent of the second exemplary embodiment, even when the lesion is grown, infiltration of the grown lesion through a hole formed in the stent can be prevented and thus the stent can be easily removed at any time. According to the second embodiment, the objects of the present invention can be achieved with a more simple structure.

### ADVANTAGEOUS EFFECTS

By providing a structure that can prevent a grown lesion infiltrating inside the extending portion of the covering unit when the stent is inserted in a lumen of the human body as described above, the stent can be easily removed without performing a surgical operation. In addition, since the covering unit pharmacologically acts on the lesion, such as cancer, the growth of the lesion can be suppressed and thus the installing state of the stent can be maintained. Therefore, the stent inserted in the lumen of the human body can be easily removed from the lumen.

### INDUSTRIAL APPLICABILITY

The stent of the present invention can be used as a medical instrument that can be used when there are abnormalities in a blood vessel, a gastrointestinal tract, a biliary tract, or the like.

1. A stent comprising:
an elastic body unit that is formed in a cylindrical shape and has an elastic force; and
a covering unit that is combined with the elastic body unit to prevent a
tissue of a human body from infiltrating inside the elastic body unit,
wherein, the covering unit comprises an extending portion that is separated from an end portion of the elastic body unit, and
a connecting string is coupled to the end portion of the elastic body unit to shrink the elastic body unit.

2. The stent of claim 1, wherein the extending portion extends past the end portion of the elastic body unit.

3. The stent of claim 1, wherein the elastic body unit is formed by twisting an elastic member having an elastic force in a mesh structure.

4. The stent of claim 1, wherein the covering unit and the extending portion are formed of a synthetic resin material or a highly polymerized compound.

5. The stent of claim 1, wherein the extending portion is provided on each of both ends of the covering unit.

6. A stent comprising:
an elastic body unit that is formed in a cylindrical shape and has an elastic force;
a covering unit combined with the elastic body unit; and
a connecting string that is coupled to the end portion of the elastic body unit to shrink the elastic body unit,
wherein, an outer circumference of the covering unit is coated with a coating layer formed of a lesion growth suppressing material or the covering unit contains a lesion growth suppressing material.

7. The stent of claim 6, wherein the lesion growth suppressing material includes paclitaxel or docetaxel.
